(19) United States
(12) Patent Application Publication
(10) Pub. No.: US 2024/0270670 A1
(43) Pub. Date: Aug. 15, 2024

(54) PROCESS FOR PARTIAL CONDENSATION OF AN OXYGENATE MIXTURE

(71) Applicant: Topsoe A/S, Kgs. Lyngby (DK)

(72) Inventors: Christian Mårup OSMUNDSEN, Gentofte (DK); Lars Saaby PEDERSEN, Farum (DK)

(73) Assignee: Topsoe A/S, Kgs. Lyngby (DK)

(21) Appl. No.: 18/289,966

(22) PCT Filed: May 27, 2022
(86) PCT No.: PCT/EP2022/064449

§ 371 Date: Nov. 8, 2023

(30) Foreign Application Priority Data
May 28, 2021 (EP) 21176507.8

(51) Int. Cl. C07C 45/45; B01D 5/00

(52) U.S. Cl. CPC C07C 45/45; B01D 5/0057

(57) ABSTRACT

There is provided a process for the partial condensation of an oxygenate mixture, the process comprising the steps of (a) providing a vapour phase oxygenate mixture obtained from fragmentation of an aqueous solution of carbohydrates; and (b) performing on the vapour phase oxygenate mixture a partial condensation to provide (i) a partial condensation condensate wherein the mass fraction of water in the partial condensation condensate is from 0.02 to 0.3 and wherein the mass ratio of glycolaldehyde to formaldehyde in the partial condensation condensate is increased relative to the mass ratio of glycolaldehyde to formaldehyde in the vapour phase oxygenate mixture; and (ii) a partial condensation vapour phase.

### FIELD OF THE INVENTION

This present invention relates to a process for the partial condensation of an oxygenate mixture to provide a condensate having an increased glycolaldehyde to formaldehyde ratio compared to the glycolaldehyde to formaldehyde ratio of the oxygenate mixture.

### BACKGROUND OF THE INVENTION

Biomass is of particular interest as a raw material due to its potential for supplementing and possibly replacing petroleum as a feedstock for the preparation of commercial chemicals. In recent years, various technologies for exploiting biomass have been investigated.

Carbohydrates represent a large fraction of biomass, and various strategies for their efficient use as a feedstock for the preparation of commercial chemicals are being established. These strategies include various fermentation-based processes, pyrolysis, and other processes, such as hydrogenolysis, hydroformylation or acid catalyzed dehydration.

Examples of chemicals produced from biomass include: substitute natural gas, biofuels, such as ethanol and bio-diesel, food browning materials, and commercial chemicals, such as diols (ethylene glycol and propylene glycol), acids (lactic acid, acrylic acid, and levulinic acid) and a wide range of other important chemical intermediates (epichlorohydrin, isoprene, furfural, and synthesis gas).

Accordingly, new uses of C1-C3 oxygenate products are being developed and an increasing demand for those products are expected. Such oxygenate products may e.g. be used for producing ethylene glycol and propylene glycol by subjecting the oxygenate product to hydrogenation (see, e.g., WO 2016/001169) or for scavenging hydrogen sulphide as described in WO 2017/064267. However, many other uses may be envisaged.

Upon fragmentation of carbohydrates, compositions consisting primarily of C1-C3 oxygenates are formed. The primary C1 oxygenate is formaldehyde, which is undesirable in many products because it is highly toxic/carcinogenic, and has been shown to act as a catalyst poison (see US 2016/002137). The primary C2 oxygenate is glycolaldehyde, which is a desired product as it may be converted to useful chemicals, such as ethylene glycol, glycolic acid and methylvinylglycolate. Oxygenate mixtures produced from the fragmentation of carbohydrates are useful in a number of different applications, where the toxicity of formaldehyde may be a problem. Preparation of a formaldehyde free, or depleted, composition is therefore highly desirable. US 2016/002137 discusses a method for removing formaldehyde by reactive distillation; however, this method adds additional process steps.

In typical known processes reported in the literature on pyrolysis of aqueous solutions of carbohydrates such as sugar and starches, the gas stream from a thermal cracker is condensed to produce a single liquid product. This is performed to ensure complete condensation and no loss of product, see for example U.S. Pat. Nos. 5,393,542 and 7,094,932. The condensed product is either then limited as to its application due to the content of formaldehyde, or must be subject to purification such as fractionation to remove formaldehyde to acceptable levels. Such a separation is particularly difficult with oxygenate mixtures prepared from aqueous solutions. This is because water is carried into the oxygenate mixture and although formaldehyde has a boiling point of −19° C., it rapidly reacts with water to form methylene glycol, which has a boiling point of 194° C. This methylene glycol is then difficult to separate from the desirable glycolaldehyde.

It would thus be desirable to provide an oxygenate mixture obtained from fragmentation of an aqueous solution of carbohydrates wherein formaldehyde is present at acceptable levels but without the need to perform a separate purification step.

### SUMMARY OF THE INVENTION

In one aspect there is provided a process for the partial condensation of an oxygenate mixture, the process comprising the steps of
(a) providing a vapour phase oxygenate mixture obtained from fragmentation of an aqueous solution of carbohydrates; and
(b) performing on the vapour phase oxygenate mixture a partial condensation to provide
   (i) a partial condensation condensate wherein the mass fraction of water in the partial condensation condensate is from 0.02 to 0.3 and wherein the mass ratio of glycolaldehyde to formaldehyde in the partial condensation condensate is increased relative to the mass ratio of glycolaldehyde to formaldehyde in the vapour phase oxygenate mixture; and
   (ii) a partial condensation vapour phase.

In one aspect there is provided a composition prepared by a process comprising the steps of
(a) providing a vapour phase oxygenate mixture obtained from fragmentation of an aqueous solution of carbohydrates; and
(b) performing on the vapour phase oxygenate mixture a partial condensation to provide
   (i) a partial condensation condensate wherein the mass fraction of water in the partial condensation condensate is from 0.02 to 0.3 and wherein the mass ratio of glycolaldehyde to formaldehyde in the partial condensation condensate is increased relative to the mass ratio of glycolaldehyde to formaldehyde in the vapour phase oxygenate mixture; and
   (ii) a partial condensation vapour phase.

In one aspect there is provided a condensate prepared by a process comprising the steps of
(a) providing a vapour phase oxygenate mixture obtained from fragmentation of an aqueous solution of carbohydrates; and
(b) performing on the vapour phase oxygenate mixture a partial condensation to provide
   (i) a partial condensation condensate wherein the mass fraction of water in the partial condensation condensate is from 0.02 to 0.3 and wherein the mass ratio of glycolaldehyde to formaldehyde in the partial condensation condensate is increased relative to the mass ratio of glycolaldehyde to formaldehyde in the vapour phase oxygenate mixture; and
   (ii) a partial condensation vapour phase.

### DETAILED DESCRIPTION OF THE INVENTION

As discussed herein, in one aspect there is provided a process for the partial condensation of an oxygenate mixture, the process comprising the steps of
(a) providing a vapour phase oxygenate mixture obtained from fragmentation of an aqueous solution of carbohydrates; and
(b) performing on the vapour phase oxygenate mixture a partial condensation to provide
   (i) a partial condensation condensate wherein the mass fraction of water in the partial condensation condensate is from 0.02 to 0.3 and wherein the mass ratio of glycolaldehyde to formaldehyde in the partial condensation condensate is increased relative to the mass ratio of glycolaldehyde to formaldehyde in the vapour phase oxygenate mixture; and
   (ii) a partial condensation vapour phase.

Upon fragmentation of carbohydrates, a composition consisting primarily of C1-C3 oxygenates is formed. Besides the main product, namely the C-2 oxygenate glycolaldehyde, the product obtained by fragmentation (e.g. thermolysis) of carbohydrates also contains significant amounts of C-1 and C-3 oxygenates as well as larger molecules. The presence of the C-1 oxygenate, formaldehyde, is often undesired and for many applications formaldehyde removal is necessary. The presence of the larger and heavier molecules in the condensate of the pyrolysis product is also undesired. Typically, significant resources may be spent on fractionation by distillation of a condensed oxygenate mixture, such as a glucose based pyrolysis product. This separation may produce an oxygenate syrup rich in glycolaldehyde and free of high-boiling byproducts but is associated with an unacceptable yield loss.

We have found that by fractional condensation of the pyrolysis vapour phase after it leaves the fragmentation stage as a vapour, one may provide an efficient process to obtain a product fraction depleted in C-1 oxygenates, while having a good yield of glycolaldehyde.

We have found that removal of energy from the vapour phase in a controlled manner, allows for partial condensation such that only part of the vapour phase is condensed (vapour to liquid phase transfer) forming a liquid condensate. Hence the product of a partial condensation is a partial condensation vapour phase enriched in the lighter components (such as formaldehyde) and a partial condensation condensate, which is a liquid phase, enriched in the heavier, condensed components and in particular glycolaldehyde.

It is surprising that formaldehyde is concentrated in the vapor phase of the partial condensation process: Although the boiling point of formaldehyde is −19° C., it is known to rapidly react with water to form methylene glycol (boiling point: 194° C.), which has a higher boiling point than glycolaldehyde (boiling point: ≈140° C.). Indeed, great care is normally taken if this reaction is to be avoided, e.g. low contact time condensers. We have surprisingly found that for this process, no such special care is required The present invention may utilise a single partial condensation. Optionally, multiple partial condensations may be carried out in series. For example, a series of condensates of different compositions may be obtained. The numbers of condensers and the cut can be adjusted for the given process. For example, for condensation of a glucose based pyrolysis product three stages could be provided, where the high-boiling components are removed as a first partial condensation condensate, a fraction rich in C-2 oxygenates are recovered as the second partial condensation condensate, and the C-1 oxygenates and water is obtained in the third and final condensation stage.

The process provides for the recovery of a C-2 oxygenate fraction with better properties (higher purity) and in higher yield than what can be obtained by fractionation of a totally condensed pyrolysis product mix.

We have identified that by performing a partial condensation a condensate can be obtained, which is significantly depleted in formaldehyde. In the examples of the present application we have provided a system in which a glycolaldehyde to formaldehyde mass ratio of 26 may be achieved, while still collecting 93% of the produced glycolaldehyde. Increasing the condenser temperature increases the glycolaldehyde to formaldehyde mass ratio to 65, at the expense of glycolaldehyde yield (57% obtained in the condensate). It should however be noted that for both experiments, addition of a secondary condenser allows for the capture of the remaining glycolaldehyde without any significant loss, compared to a process consisting of only one total condensation.

It is highly surprising that such a good separation between glycolaldehyde and formaldehyde is possible. As discussed above, although formaldehyde has a boiling point of −19° C., it is known to rapidly react with water to form methylene glycol, which has a boiling point of 194° C., thus it would be expected that formaldehyde would be collected in the primary condenser. In fact, great pains are normally taken to keep the residence time low during partial condensation of formaldehyde/water mixtures, to avoid the reaction of water and formaldehyde. We have found that in the present process no such efforts were required, and in fact no significant effect of residence time has been observed. Without being bound by theory it is believed that this may be because “free” water is kept to a very low level (and thus the reaction with formaldehyde is prevented) as the condensed glycolaldehyde is able to react with water to form a hydrate. Thus although use of partial condensation of pyrolysis vapors produced by thermolysis of solid biomass is known, it was highly surprising that such a configuration could address the problems of vapor produced by fragmentation of an aqueous solutions of carbohydrates, namely the problem of methylene glycol formation.

A number of references teach partial condensation of pyrolysis vapors produced by thermolysis of solid biomass. For example Papari, S., Hawboldt, K. Fuel Processing Technology 180 (2018) 1-13 is a review article on condensing systems for biomass pyrolysis processes which focuses on processes for conversion of lignocellulose/woody biomass to pyrolysis oil for use as fuel. Siriwardhana, M. Renewable Energy 152 (2020) 1121-1128 is a more recent paper focusing on fractional condensation of pyrolysis vapor produced from lignocellulose for stabilization of the bio oil fraction. Pollard, A. S. et al., Journal of analytical and Applied Pyrolysis 93 (2012) 129-138 gives a description of a staged fractionation process downstream to a fluid bed fast pyrolysis unit. Westerhof, R. J. M et al., Energy Fuels 2011, 25, 1817-1829 provides a detailed description of a two stage counter current spray column system for fractional condensation of pyrolysis vapor obtained by fast pyrolysis of soft wood. Tumbalam Gooty, A., “Fractional Condensation of Bio-Oil Vapors” (2012). Electronic Thesis and Dissertation Repository. Paper 979. The University of Western Ontario contains a number of references to other publications on condensation of bio-oil including A. V. Bridgwater, G. V. C. Peacocke, Renewable and Sustainable Energy Reviews 4 (2000) 1-73, and J. F. Waker, Formaldehyde, Reinhold, 1964 relating to partial condensation for recovery of formaldehyde from aqueous gas phase. EP2231817 describes a two stage condensing of pyrolysis gas in which the process focusses on short cooling time in the condensers. U.S. Pat. No. 8,476,480 describes the partial condensation of bio oil. US20110139597 describes water removal from thermochemically converted biomass by partial condensations. U.S. Pat. No. 2,675,346 describes purification of formaldehyde by partial condensation using a system with multiple partial condensation steps. However, none of these references hint at the surprising findings of the present invention. EP3786145 relates to a process for large scale and energy efficient production of oxygenates from sugar in which a sugar feedstock is introduced into a thermolytic fragmentation reactor comprising a fluidized stream of heat carrying particles. The heat carrying particles may be separated from the fluidized stream prior to cooling the fragmentation product and may be directed to a reheater to reheat the particles and recirculate the heated particles to the fragmentation reactor. The Example of EP3786145 describes a process similar to the present invention but contains a partial condensation condensate in which the mass fraction of water is from 0.51 to 0.6.

For ease of reference, these and further aspects of the present invention are now discussed under appropriate section headings. However, the teachings under each section are not necessarily limited to each particular section.

### Water

As discussed herein, there is provided a vapour phase oxygenate mixture obtained from fragmentation of an aqueous solution of carbohydrates. It has been found that the formation of the vapour phase oxygenate mixture obtained by fragmentation of an aqueous solution of carbohydrates may result in problems with separating formaldehyde from the oxygenate mixture. We have found that these problems may be overcome using the partial condensation process of the present invention. In particular, we found that when keeping the water concentration in the partial condensation condensate relatively low, the tendency of formaldehyde to condense may be greatly reduced. Instead, formaldehyde remains in the vapour phase and could thus be removed from the other (heavier) oxygenates. It is thus desirable that the partial condensation condensate has a low concentration of water. When referring to “a low concentration of water” it is to be understood that some water may be bound as a glycolaldehyde hydrate. The water concentration of the partial condensation condensate may be determined by the Karl-Fischer titration method. It will be appreciated by one skilled in the art that the mass fraction of water in the partial condensation condensate may be controlled by controlling the process parameters. These process parameters will depend on, among other things, the source material of the vapour phase oxygenate material, and it will be understood that multiple process parameters may be inter dependent i.e. changing one parameter will modify the optimum for one or more other process parameters. These process parameters may be readily determined and controlled by one skilled in the art.

The mass fraction of water in the partial condensation condensate is from 0.02 to 0.3. In one aspect, the mass fraction of water in the partial condensation condensate is from 0.03 to 0.3. In one aspect, the mass fraction of water in the partial condensation condensate is from 0.04 to 0.3. In one aspect, the mass fraction of water in the partial condensation condensate is from 0.05 to 0.3. In one aspect, the mass fraction of water in the partial condensation condensate is from 0.02 to 0.2. In one aspect, the mass fraction of water in the partial condensation condensate is from 0.03 to 0.2. In one aspect, the mass fraction of water in the partial condensation condensate is from 0.04 to 0.2. In one aspect, the mass fraction of water in the partial condensation condensate is from 0.05 to 0.2. In one aspect, the mass fraction of water in the partial condensation condensate is from 0.02 to 0.15. In one aspect, the mass fraction of water in the partial condensation condensate is from 0.03 to 0.15. In one aspect, the mass fraction of water in the partial condensation condensate is from 0.04 to 0.15. In one aspect, the mass fraction of water in the partial condensation condensate is from 0.05 to 0.15. In one aspect, the mass fraction of water in the partial condensation condensate is from 0.02 to 0.1. In one aspect, the mass fraction of water in the partial condensation condensate is from 0.03 to 0.1. In one aspect, the mass fraction of water in the partial condensation condensate is from 0.04 to 0.1. In one aspect, the mass fraction of water in the partial condensation condensate is from 0.05 to 0.1.

### Partial Condensation Condensate

As discussed herein, the mass ratio of glycolaldehyde to formaldehyde in the partial condensation condensate is increased relative to the mass ratio of glycolaldehyde to formaldehyde in the vapour phase oxygenate mixture. In one aspect, not only is the mass fraction of glycolaldehyde in the partial condensation condensate increased with respect to the mass fraction of glycolaldehyde in the oxygenate mixture, but the mass fraction of formaldehyde in the partial condensation condensate is decreased with respect to the mass fraction of formaldehyde in the oxygenate mixture. Thus, in one aspect the partial condensation condensate has a mass fraction of formaldehyde decreased with respect to the mass fraction of formaldehyde in the oxygenate mixture.

As will be appreciated by one skilled in the art, the vapour phase oxygenate mixture will have a ratio of glycolaldehyde to formaldehyde which may vary depending on the carbohydrate feed and the conditions of the fragmentation. In one aspect, the vapour phase oxygenate mixture has a mass ratio of glycolaldehyde to formaldehyde of 2:1 to 18:1. In one aspect, the vapour phase oxygenate mixture has a mass ratio of glycolaldehyde to formaldehyde of 3:1 to 18:1. In one aspect, the vapour phase oxygenate mixture has a mass ratio of glycolaldehyde to formaldehyde of 4:1 to 18:1. In one aspect, the vapour phase oxygenate mixture has a mass ratio of glycolaldehyde to formaldehyde of 5:1 to 18:1. In one aspect, the vapour phase oxygenate mixture has a mass ratio of glycolaldehyde to formaldehyde of 2:1 to 15:1. In one aspect, the vapour phase oxygenate mixture has a mass ratio of glycolaldehyde to formaldehyde of 3:1 to 15:1. In one aspect, the vapour phase oxygenate mixture has a mass ratio of glycolaldehyde to formaldehyde of 4:1 to 15:1. In one aspect, the vapour phase oxygenate mixture has a mass ratio of glycolaldehyde to formaldehyde of 5:1 to 15:1. In one aspect, the vapour phase oxygenate mixture has a mass ratio of glycolaldehyde to formaldehyde of 2:1 to 10:1. In one aspect, the vapour phase oxygenate mixture has a mass ratio of glycolaldehyde to formaldehyde of 3:1 to 10:1. In one aspect, the vapour phase oxygenate mixture has a mass ratio of glycolaldehyde to formaldehyde of 4:1 to 10:1. In one aspect, the vapour phase oxygenate mixture has a mass ratio of glycolaldehyde to formaldehyde of 5:1 to 10:1.

As discussed above, in one aspect the partial condensation condensate has a mass fraction of formaldehyde decreased with respect to the mass fraction of formaldehyde in the oxygenate mixture. In one aspect, the partial condensation condensate has a mass ratio of glycolaldehyde to formaldehyde of at least 5:1. In one aspect, the partial condensation condensate has a mass ratio of glycolaldehyde to formaldehyde of at least 10:1. In one aspect, the partial condensation condensate has a mass ratio of glycolaldehyde to formaldehyde of at least 15:1. In one aspect, the partial condensation condensate has a mass ratio of glycolaldehyde to formaldehyde of at least 20:1. In one aspect, the partial condensation condensate has a mass ratio of glycolaldehyde to formaldehyde of at least 30:1. In one aspect, the partial condensation condensate has a mass ratio of glycolaldehyde to formaldehyde of at least 40:1. In one aspect, the partial condensation condensate has a mass ratio of glycolaldehyde to formaldehyde of at least 50:1. In one aspect, the partial condensation condensate has a mass ratio of glycolaldehyde to formaldehyde of at least 60:1. In one aspect, the partial condensation condensate has a mass ratio of glycolaldehyde to formaldehyde of at least 70:1. In one aspect, the partial condensation condensate has a mass ratio of glycolaldehyde to formaldehyde of at least 80:1. In one aspect, the partial condensation condensate has a mass ratio of glycolaldehyde to formaldehyde of at least 90:1. In one aspect, the partial condensation condensate has a mass ratio of glycolaldehyde to formaldehyde of at least 100:1.

In one aspect, the partial condensation condensate has a mass ratio of glycolaldehyde to formaldehyde of no greater than 500:1. In one aspect, the partial condensation condensate has a mass ratio of glycolaldehyde to formaldehyde of no greater than 400:1. In one aspect, the partial condensation condensate has a mass ratio of glycolaldehyde to formaldehyde of no greater than 300:1. In one aspect, the partial condensation condensate has a mass ratio of glycolaldehyde to formaldehyde of no greater than 200:1. In one aspect, the partial condensation condensate has a mass ratio of glycolaldehyde to formaldehyde of no greater than 180:1. In one aspect, the partial condensation condensate has a mass ratio of glycolaldehyde to formaldehyde of no greater than 160:1. In one aspect, the partial condensation condensate has a mass ratio of glycolaldehyde to formaldehyde of no greater than 150:1. In one aspect, the partial condensation condensate has a mass ratio of glycolaldehyde to formaldehyde of no greater than 140:1. In one aspect, the partial condensation condensate has a mass ratio of glycolaldehyde to formaldehyde of no greater than 130:1. In one aspect, the partial condensation condensate has a mass ratio of glycolaldehyde to formaldehyde of no greater than 120:1. In one aspect, the partial condensation condensate has a mass ratio of glycolaldehyde to formaldehyde of no greater than 110:1. In one aspect, the partial condensation condensate has a mass ratio of glycolaldehyde to formaldehyde of no greater than 100:1.

In one aspect, the partial condensation condensate has a mass ratio of glycolaldehyde to formaldehyde of from 10:1 to 200:1. In one aspect, the partial condensation condensate has a mass ratio of glycolaldehyde to formaldehyde of from 10:1 to 180:1. In one aspect, the partial condensation condensate has a mass ratio of glycolaldehyde to formaldehyde of from 10:1 to 160:1. In one aspect, the partial condensation condensate has a mass ratio of glycolaldehyde to formaldehyde of from 10:1 to 150:1. In one aspect, the partial condensation condensate has a mass ratio of glycolaldehyde to formaldehyde of from 10:1 to 140:1. In one aspect, the partial condensation condensate has a mass ratio of glycolaldehyde to formaldehyde of from 10:1 to 130:1. In one aspect, the partial condensation condensate has a mass ratio of glycolaldehyde to formaldehyde of from 10:1 to 120:1. In one aspect, the partial condensation condensate has a mass ratio of glycolaldehyde to formaldehyde of from 10:1 to 110:1. In one aspect, the partial condensation condensate has a mass ratio of glycolaldehyde to formaldehyde of from 10:1 to 100:1.

In one aspect, the partial condensation condensate has a mass ratio of glycolaldehyde to formaldehyde of from 30:1 to 200:1. In one aspect, the partial condensation condensate has a mass ratio of glycolaldehyde to formaldehyde of from 30:1 to 180:1. In one aspect, the partial condensation condensate has a mass ratio of glycolaldehyde to formaldehyde of from 30:1 to 160:1. In one aspect, the partial condensation condensate has a mass ratio of glycolaldehyde to formaldehyde of from 30:1 to 150:1. In one aspect, the partial condensation condensate has a mass ratio of glycolaldehyde to formaldehyde of from 30:1 to 140:1. In one aspect, the partial condensation condensate has a mass ratio of glycolaldehyde to formaldehyde of from 30:1 to 130:1. In one aspect, the partial condensation condensate has a mass ratio of glycolaldehyde to formaldehyde of from 30:1 to 120:1. In one aspect, the partial condensation condensate has a mass ratio of glycolaldehyde to formaldehyde of from 30:1 to 130:1. In one aspect, the partial condensation condensate has a mass ratio of glycolaldehyde to formaldehyde of from 30:1 to 100:1.

The mass ratio of glycolaldehyde to formaldehyde in the partial condensation condensate is increased relative to the mass ratio of glycolaldehyde to formaldehyde in the vapour phase oxygenate mixture. In one aspect, the mass ratio of glycolaldehyde to formaldehyde in the partial condensation condensate is increased at least 2 fold relative to the mass ratio of glycolaldehyde to formaldehyde in the vapour phase oxygenate mixture. For example, if the mass ratio of glycolaldehyde to formaldehyde in the partial condensation condensate is 2:1, the mass ratio of glycolaldehyde to formaldehyde in the vapour phase oxygenate mixture would be at least 4:1. In one aspect, the mass ratio of glycolaldehyde to formaldehyde in the partial condensation condensate is increased at least 3 fold relative to the mass ratio of glycolaldehyde to formaldehyde in the vapour phase oxygenate mixture. In one aspect, the mass ratio of glycolaldehyde to formaldehyde in the partial condensation condensate is increased at least 4-fold relative to the mass ratio of glycolaldehyde to formaldehyde in the vapour phase oxygenate mixture. In one aspect, the mass ratio of glycolaldehyde to formaldehyde in the partial condensation condensate is increased at least 5-fold relative to the mass ratio of glycolaldehyde to formaldehyde in the vapour phase oxygenate mixture. In one aspect, the mass ratio of glycolaldehyde to formaldehyde in the partial condensation condensate is increased at least 10-fold relative to the mass ratio of glycolaldehyde to formaldehyde in the vapour phase oxygenate mixture. In one aspect, the mass ratio of glycolaldehyde to formaldehyde in the partial condensation condensate is increased at least 15-fold relative to the mass ratio of glycolaldehyde to formaldehyde in the vapour phase oxygenate mixture. In one aspect, the mass ratio of glycolaldehyde to formaldehyde in the partial condensation condensate is increased at least 20-fold relative to the mass ratio of glycolaldehyde to formaldehyde in the vapour phase oxygenate mixture.

As discussed herein, wherein the mass ratio of glycolaldehyde to formaldehyde in the partial condensation condensate is increased relative to the mass ratio of glycolaldehyde to formaldehyde in the vapour phase oxygenate mixture. Any level of increase will satisfy this requirement. However, in some aspects, it is desirable to collect in the partial condensation condensate particular proportions of the total amount of the glycolaldehyde which is present in the vapour phase oxygenate mixture. In one aspect, the partial condensation condensate contains at least 30 wt. % of the glycolaldehyde present in the vapour phase oxygenate mixture. In one aspect, the partial condensation condensate contains at least 40 wt. % of the glycolaldehyde present in the vapour phase oxygenate mixture. In one aspect, the partial condensation condensate contains at least 50 wt. % of the glycolaldehyde present in the vapour phase oxygenate mixture. In one aspect, the partial condensation condensate contains at least 60 wt. % of the glycolaldehyde present in the vapour phase oxygenate mixture. In one aspect, the partial condensation condensate contains at least 70 wt. % of the glycolaldehyde present in the vapour phase oxygenate mixture. In one aspect, the partial condensation condensate contains at least 80 wt. % of the glycolaldehyde present in the vapour phase oxygenate mixture. In one aspect, the partial condensation condensate contains at least 90 wt. % of the glycolaldehyde present in the vapour phase oxygenate mixture. In one aspect, the partial condensation condensate contains at least 95 wt. % of the glycolaldehyde present in the vapour phase oxygenate mixture.

### Condensation

As discussed herein the present process requires step b) performing on the vapour phase oxygenate mixture a partial condensation to provide (i) a partial condensation condensate wherein the mass ratio of glycolaldehyde to formaldehyde in the partial condensation condensate is increased relative to the mass ratio of glycolaldehyde to formaldehyde in the vapour phase oxygenate mixture; and (ii) a partial condensation vapour phase. The step (b) may be preceded by one or more condensation steps. The step (b) may be followed by one or more condensation steps. For example, as discussed herein, multiple fractional condensations may be carried out in series. For example, a series of condensates of different compositions may be obtained. The numbers of condensers and the cut can be adjusted for the given process. For example, for fractionation of a glucose based pyrolysis product three stages could be provided, where the high-boiling components are removed as a first partial condensation condensate, a fraction rich in C-2 oxygenates are recovered as the second partial condensation condensate, and the C-1 oxygenates and water is obtained in the third and final partial condensation stage.

In one aspect, the present process comprises a further step of (c) performing on the partial condensation vapour phase of step (b) a second condensation step to provide a second condensate.

In one aspect, the second condensation step is a total condensation of the partial condensation vapour phase of step (b). In one aspect, the second condensation step is a partial condensation of the partial condensation vapour phase of step (b).

As will be appreciated by one skilled in the art, if the second condensation step is a total condensation of the partial condensation vapour phase of step (b), the second condensate may have a mass fraction of formaldehyde increased with respect to the mass fraction of formaldehyde in the vapour phase oxygenate mixture.

As will be appreciated by one skilled in the art, if the second condensation step is a total condensation of the partial condensation vapour phase of step (b), the mass ratio of glycolaldehyde to formaldehyde in the second condensate is decreased relative to the mass ratio of glycolaldehyde to formaldehyde in the vapour phase oxygenate mixture.

### Vapour Phase Oxygenate Mixture

The “vapour phase oxygenate mixture” may also be referred to as a “pyrolysis product”, a “pyrolysis product mix”, a “pyrolysis vapour phase” etc. The present process relates to the partial condensation of an oxygenate mixture which is obtained from fragmentation of an aqueous solution of carbohydrates. In one aspect, the fragmentation step is incorporated in the present process. In one aspect, the present invention includes the step of fragmentation of an aqueous solution of carbohydrates to provide the vapour phase oxygenate mixture of step (a).

Thus in one aspect there is provided a process for the partial condensation of an oxygenate mixture, the process comprising the steps of
(a) fragmentation of an aqueous solution of carbohydrates to provide a vapour phase oxygenate mixture; and
(b) performing on the vapour phase oxygenate mixture a partial condensation to provide (i) a partial condensation condensate wherein the mass ratio of glycolaldehyde to formaldehyde in the partial condensation condensate is increased relative to the mass ratio of glycolaldehyde to formaldehyde in the vapour phase oxygenate mixture; and (ii) a partial condensation vapour phase.

The fragmentation of the aqueous solution of carbohydrates may be achieved by any suitable means. In one aspect, the fragmentation of the aqueous solution of carbohydrates is thermolytic fragmentation. In one aspect, the fragmentation of the aqueous solution of carbohydrates is pyrolysis. The fragmentation of the aqueous solution of carbohydrates may be performed by any suitable process. In one aspect the fragmentation of the aqueous solution of carbohydrates is performed as described in WO2020/016209.

The carbohydrates of the aqueous solution may be any suitable carbohydrates. In one aspect, the carbohydrates are selected from monosaccharides, disaccharides and mixtures thereof. In one aspect, the carbohydrates are at least monosaccharides. In one aspect, the carbohydrate is at least glucose.

In one aspect, the carbohydrates of the aqueous solution of carbohydrates comprise at least 20 wt. % monosaccharides based on the total amount of carbohydrates. In one aspect, the carbohydrates of the aqueous solution of carbohydrates comprise at least 30 wt. % monosaccharides based on the total amount of carbohydrates. In one aspect, the carbohydrates of the aqueous solution of carbohydrates comprise at least 40 wt. % monosaccharides based on the total amount of carbohydrates. In one aspect, the carbohydrates of the aqueous solution of carbohydrates comprise at least 50 wt. % monosaccharides based on the total amount of carbohydrates. In one aspect, the carbohydrates of the aqueous solution of carbohydrates comprise at least 60 wt. % monosaccharides based on the total amount of carbohydrates. In one aspect, the carbohydrates of the aqueous solution of carbohydrates comprise at least 70 wt. % monosaccharides based on the total amount of carbohydrates. In one aspect, the carbohydrates of the aqueous solution of carbohydrates comprise at least 80 wt. % monosaccharides based on the total amount of carbohydrates. In one aspect, the carbohydrates of the aqueous solution of carbohydrates comprise at least 90 wt. % monosaccharides based on the total amount of carbohydrates. In one aspect, the carbohydrates of the aqueous solution of carbohydrates comprise at least 95 wt. % monosaccharides based on the total amount of carbohydrates.

In one aspect, the carbohydrates of the aqueous solution of carbohydrates comprise at least 20 wt. % glucose based on the total amount of carbohydrates. In one aspect, the carbohydrates of the aqueous solution of carbohydrates comprise at least 30 wt. % glucose based on the total amount of carbohydrates. In one aspect, the carbohydrates of the aqueous solution of carbohydrates comprise at least 40 wt. % glucose based on the total amount of carbohydrates. In one aspect, the carbohydrates of the aqueous solution of carbohydrates comprise at least 50 wt. % glucose based on the total amount of carbohydrates. In one aspect, the carbohydrates of the aqueous solution of carbohydrates comprise at least 60 wt. % glucose based on the total amount of carbohydrates. In one aspect, the carbohydrates of the aqueous solution of carbohydrates comprise at least 70 wt. % glucose based on the total amount of carbohydrates. In one aspect, the carbohydrates of the aqueous solution of carbohydrates comprise at least 80 wt. % glucose based on the total amount of carbohydrates. In one aspect, the carbohydrates of the aqueous solution of carbohydrates comprise at least 90 wt. % glucose based on the total amount of carbohydrates. In one aspect, the carbohydrates of the aqueous solution of carbohydrates comprise at least 95 wt. % glucose based on the total amount of carbohydrates.

The aqueous solution of carbohydrates may comprise carbohydrates in any suitable amount. In one aspect, the aqueous solution of carbohydrates comprises carbohydrates in an amount of at least 10 wt. % based on the aqueous solution. In one aspect, the aqueous solution of carbohydrates comprises carbohydrates in an amount of at least 20 wt. % based on the aqueous solution. In one aspect, the aqueous solution of carbohydrates comprises carbohydrates in an amount of at least 30 wt. % based on the aqueous solution. In one aspect, the aqueous solution of carbohydrates comprises carbohydrates in an amount of at least 40 wt. % based on the aqueous solution. In one aspect, the aqueous solution of carbohydrates comprises carbohydrates in an amount of at least 50 wt. % based on the aqueous solution. In one aspect, the aqueous solution of carbohydrates comprises carbohydrates in an amount of at least 60 wt. % based on the aqueous solution. In one aspect, the aqueous solution of carbohydrates comprise glucose in an amount of at least 10 wt. % based on the aqueous solution. In one aspect, the aqueous solution of carbohydrates comprise glucose in an amount of at least 20 wt. % based on the aqueous solution. In one aspect, the aqueous solution of carbohydrates comprise glucose in an amount of at least 30 wt. % based on the aqueous solution. In one aspect, the aqueous solution of carbohydrates comprise glucose in an amount of at least 40 wt. % based on the aqueous solution. In one aspect, the aqueous solution of carbohydrates comprise glucose in an amount of at least 50 wt. % based on the aqueous solution. In one aspect, the aqueous solution of carbohydrates comprise glucose in an amount of at least 60 wt. % based on the aqueous solution.

The process parameters required to perform the partial condensation of the present invention can be readily determined by one skilled in the art. Key parameters are the composition of the vapour phase oxygenate mixture, the partial condensation temperature and the partial condensation pressure. In general, the composition of the vapour phase oxygenate mixture will be defined by the fragmentation process. Similarly, the pressure of the partial condensation will typically be defined by requirements of the upstream process. Thus, the temperature of the partial condensation is the parameter typically controlled to achieve the required separation. If the pressure is not defined by upstream process considerations, and can be freely controlled, in principle this may be used as the controlling parameter instead (at a fixed temperature). However for most practical applications, controlling the temperature of the partial condensation will be more appropriate. In any case, the considerations for using the pressure as controlling parameter will be identical to those outlined below for temperature.

In the present invention, we have found that to achieve a good separation of formaldehyde and glycolaldehyde, a suitable water content in the partial condensation condensate should be achieved. As described, this suitable water content will typically be 2 to 20 wt. %. The water content of the partial condensation condensate can easily be determined using known analysis techniques, such as Karl-Fischer titration. If the water content of the first condensate is below the optimal value, the temperature of the partial condensation condenser should be decreased, and similarly if the water content is higher than optimal, the temperature of the partial condensation condenser should be increased.

In one aspect, the partial condensation condensate is condensed at a temperature of from 0 to 150° C. In one aspect, the partial condensation condensate is condensed at a temperature of from 10 to 150° C. In one aspect, the partial condensation condensate is condensed at a temperature of from 20 to 150° C. In one aspect, the partial condensation condensate is condensed at a temperature of from 30 to 150° C. In one aspect, the partial condensation condensate is condensed at a temperature of from 30 to 130° C. In one aspect, the partial condensation condensate is condensed at a temperature of from 0 to 90° C. In one aspect, the partial condensation condensate is condensed at a temperature of from 5 to 90° C. In one aspect, the partial condensation condensate is condensed at a temperature of from 40 to 90° C. In one aspect there is provided a process for the partial condensation of an oxygenate mixture, the process comprising the steps of
(a) providing a vapour phase oxygenate mixture obtained from fragmentation of an aqueous solution of carbohydrates; and
(b) performing on the vapour phase oxygenate mixture a partial condensation to provide
   (i) a partial condensation condensate, wherein the partial condensation condensate is condensed at a temperature of from 0 to 150° C. and
   (ii) a partial condensation vapour phase.

As discussed herein, prior to step (b) an intermediate step may be performed on the oxygenate mixture. For example, an initial partial condensation may be performed on the oxygenate mixture prior to the partial condensation step (b). In one aspect, step (b) is performed on an oxygenate mixture obtained directly from fragmentation of an aqueous solution of carbohydrates. This intermediate step may be selected, for example, depending on the composition of the oxygenate mixture obtained directly from fragmentation of an aqueous solution of carbohydrates. As will be understood by one skilled in the art, the composition of the oxygenate mixture obtained directly from fragmentation of an aqueous solution of carbohydrates will depend, among other things, on the composition of the aqueous solution of carbohydrates and the fragmentation process parameters.

### Further Process Steps

The process of the present invention may comprise one or more further steps. These one or more further steps may be before, after, or intermediate to the steps recited herein.

### Further Aspects

In one aspect there is provided a process for the partial condensation of an oxygenate mixture, the process comprising the steps of
(a) providing a vapour phase oxygenate mixture obtained from fragmentation of an aqueous solution of carbohydrates; and
(b) performing on the vapour phase oxygenate mixture a partial condensation to provide
   (i) a partial condensation condensate wherein the mass ratio of glycolaldehyde to formaldehyde in the partial condensation condensate is increased relative to the mass ratio of glycolaldehyde to formaldehyde in the vapour phase oxygenate mixture; and
   (ii) a partial condensation vapour phase.

In one aspect there is provided a composition prepared by a process comprising the steps of
(a) providing a vapour phase oxygenate mixture obtained from fragmentation of an aqueous solution of carbohydrates; and
(b) performing on the vapour phase oxygenate mixture a partial condensation to provide
   (i) a partial condensation condensate wherein the mass ratio of glycolaldehyde to formaldehyde in the partial condensation condensate is increased relative to the mass ratio of glycolaldehyde to formaldehyde in the vapour phase oxygenate mixture; and
   (ii) a partial condensation vapour phase.

In one aspect there is provided a condensate prepared by a process comprising the steps of
(a) providing a vapour phase oxygenate mixture obtained from fragmentation of an aqueous solution of carbohydrates; and
(b) performing on the vapour phase oxygenate mixture a partial condensation to provide
   (i) a partial condensation condensate wherein the mass ratio of glycolaldehyde to formaldehyde in the partial condensation condensate is increased relative to the mass ratio of glycolaldehyde to formaldehyde in the vapour phase oxygenate mixture; and
   (ii) a partial condensation vapour phase.

The invention will now be described with reference to the following non-limiting examples.

### EXAMPLES

### Example 1

Sodium silicate glass beads (100 g) was loaded in a bubbling fluid bed reactor (42 mm ID) and fluidized at a superficial gas velocity of approx. 50 cm/s. The temperature was increased to 520° C., at which point a feed of water was injected into the bed. The feed was injected through a two-fluid nozzle at a rate of 2 g/min. Once the system reached steady state, the feed was switched to a 30 wt. % aqueous solution of glucose and time set as to. The gas leaving the reactor (vapour phase oxygenate mixture) was cooled (to achieve a partial condensation) in a primary condenser, and a first condensate was collected. The temperature in the primary condenser was varied between 3 and 67° C. The vapour phase leaving the primary condenser was sent to a secondary condenser held at 1° C. to condense the remaining condensable components as a second condensate. The concentrations of oxygenates in the condensates were determined by HPLC analysis, and the yield of oxygenates calculated based on the mass of collected product. Table 1 below shows results for the first liquid condensate.

TABLE 1
Data for the analysis of first liquid condensate at various temperatures. In
these examples, the feed to the first condenser is the hot gas leaving the
reactor (vapour phase oxygenate mixture). “Mass collected” is the mass
of first condensate per mass of feed fed to the bubbling fluid bed reactor.
The amounts of glycolaldehyde and formaldehyde, respectively, are the amounts
of carbon present in glycolaldehyde and formaldehyde, respectively, in the
first condensate, per total amount of carbon present in the feed fed to the
bubbling fluid bed reactor. Water is the percentage of water in the first condensate.

| | Partial | | | | |
|---|---|---|---|---|---|
| | Condensation | Mass | | | |
| | Condenser | collected | Glycolaldehyde | Formaldehyde | Water |
| Reference | temp. [° C.] | [%] | [C. %] | [C. %] | [wt. %] |
| 100-01 | 3 | 91 | 67.6 | 5.2 | 72 |
| 103-01 | 47 | 37 | 63.9 | 2.5 | 40 |
| 109-01 | 57 | 23 | 54.3 | 1.1 | 22 |
| 112-02 | 59 | 22 | 53.9 | 1.4 | 17 |
| 112-01 | 62 | 16 | 42.8 | 1.1 | 10 |
| 109-02 | 67 | 15 | 38.8 | 0.6 | 15 |
| 103-02 | 67 | 17 | 36.5 | 0.9 | 27 |

Increasing the temperature of the primary condenser from 3° C. to 47° C. led to a 52% decrease in the amount of formaldehyde collected, while only 5% less glycolaldehyde was collected. Increasing the temperature to 67° C. led to a decrease of formaldehyde of 88%, while still collecting 57% of the glycolaldehyde.

### Example 2

An aqueous solution of oxygenates, produced by the method described in example 1, was evaporated. The mixture contained 141 g/L of glycolaldehyde and 29 g/L of formaldehyde. The evaporation was performed by feeding 0.1 g/L of oxygenate solution, together with 100 NmL/min of nitrogen, into an evaporator held at 300° C. The produced gas stream (vapour phase oxygenate mixture) was directed to a primary condenser (to achieve partial condensation) maintained at a temperature from 5-90° C. to collect a first condensate. The vapour phase leaving the primary condenser was sent to a secondary condenser held at 1° C. to condense the remaining condensable components as a second condensate. The concentrations of oxygenates in the condensates were determined by HPLC analysis, and the yield of oxygenates calculated based on the mass of collected product. Table 2 below shows results for the first liquid condensate.

TABLE 2
Data for the analysis of first liquid condensate at various
temperatures. In these examples, the feed fed to the first
condenser is a vapour phase oxygenate mixture prepared by evaporation
of a liquid pyrolysis product to produce the vapour phase oxygenate
mixture. “Mass collected” is the mass of first condensate
per mass of oxygenate solution evaporated and fed to the primary
condenser. The recovery of glycolaldehyde is the percentage
of the glycolaldehyde present in the vapour phase oxygenate
mixture fed to the primary condenser, which was collected in
the first condensate. The recovery of formaldehyde is the percentage
of the formaldehyde present in the vapour phase oxygenate mixture
fed to the primary condenser, which was collected in the first
condensate.

| | Partial | | | |
|---|---|---|---|---|
| | Condensation | Mass | Recovery of | Recovery of |
| | Condenser | collected | glycolaldehyde | formaldehyde |
| Reference | temp. [° C] | [%] | [%] | [%] |
| 13-02 | 5 | 95 | 95 | 98 |
| 19-01 | 50 | 86 | 95 | 57 |
| 19-02 | 70 | 81 | 92 | 54 |
| 19-03 | 90 | 40 | 91 | 29 |

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. A process for the partial condensation of an oxygenate mixture, the process comprising the steps of
(a) providing a vapour phase oxygenate mixture obtained from fragmentation of an aqueous solution of carbohydrates; and
(b) performing on the vapour phase oxygenate mixture a partial condensation to provide
(i) a partial condensation condensate wherein the mass fraction of water in the partial condensation condensate is from 0.02 to 0.3 and wherein the mass ratio of glycolaldehyde to formaldehyde in the partial condensation condensate is increased relative to the mass ratio of glycolaldehyde to formaldehyde in the vapour phase oxygenate mixture; and
(ii) a partial condensation vapour phase.

2. A process according to claim 1 wherein the mass fraction of water in the partial condensation condensate is from 0.02 to 0.2.

3. A process according to claim 1 wherein the mass fraction of water in the partial condensation condensate is from 0.03 to 0.1.

4. A process according to claim 1 wherein the mass fraction of water in the partial condensation condensate is from 0.05 to 0.1.

5. A process according to claim 1 wherein the partial condensation condensate has a mass fraction of formaldehyde decreased with respect to the mass fraction of formaldehyde in the oxygenate mixture.

6. A process according to claim 1 wherein the vapour phase oxygenate mixture has a mass ratio of glycolaldehyde to formaldehyde of 2:1 to 18:1.

7. A process according to claim 1 wherein the partial condensation condensate has a mass ratio of glycolaldehyde to formaldehyde of at least 20:1.

8. A process according to claim 1 wherein the partial condensation condensate has a mass ratio of glycolaldehyde to formaldehyde of at least 50:1.

9. A process according to claim 1 wherein the partial condensation condensate contains at least 50 wt. % of the glycolaldehyde present in the vapour phase oxygenate mixture.

10. A process according to claim 1 wherein the partial condensation condensate contains at least 80 wt. % of the glycolaldehyde present in the vapour phase oxygenate mixture

11. A process according to claim 1 wherein the partial condensation condensate contains at least 90 wt. % of the glycolaldehyde present in the vapour phase oxygenate mixture.

12. A process according to claim 1 comprising a further step of
(c) performing on the partial condensation vapour phase mixture of step (b) a second condensation step to provide a second condensate.

13. A process according to claim 12 wherein the second condensate has a mass fraction of formaldehyde increased with respect to mass fraction of formaldehyde in the vapour phase oxygenate mixture.

14. A process according to claim 12 wherein the second condensate of the second condensation step is condensed at a temperature of from −20 to 30° C.

15. A process according to claim 12, wherein the second condensate of the second condensation step is condensed at a temperature of from −10 to 10° C.

16. A process according to claim 12 wherein the second condensation step is a partial condensation to provide a second condensate wherein the mass ratio of glycolaldehyde to formaldehyde in the second condensate is increased relative to the mass ratio of glycolaldehyde to formaldehyde in the vapour phase oxygenate mixture.

17. A process according to claim 1 comprising the step of fragmentation of the aqueous solution of carbohydrates to provide the vapour phase oxygenate mixture of step (a).

18. A process according to claim 1 wherein the fragmentation of the aqueous solution of carbohydrates is thermolytic fragmentation.

19. A process according to claim 1 wherein the carbohydrates of the aqueous solution of carbohydrates comprise at least 90 wt. % monosaccharides based on the total amount of carbohydrates.

20. A process according to claim 1 wherein step (b) is performed on an oxygenate mixture obtained directly from fragmentation of an aqueous solution of carbohydrates.

21. A process according to claim 1 wherein the partial condensation condensate is condensed at a temperature of from 0 to 150° C.

22. A process according to claim 21 wherein the partial condensation condensate is condensed at a temperature of from 0 to 90° C.
